(19) United States
(12) Patent Application Publication
(10) Pub. No.: US 2009/0024069 A1
(43) Pub. Date: Jan. 22, 2009

(54) COMPRESSION WRAP AND GARMENT

(76) Inventor: Julie Appel, Eden Prairie, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/777,722

(22) Filed: Jul. 16, 2007

(51) Int. Cl. A61F 13/12; A61F 13/00

(52) U.S. Cl. 602/74; 602/75

(57) ABSTRACT

A compression garment for use following cosmetic surgery can include elastic compression regions shaped to selectively conform to a portion of a user's body subjected to cosmetic surgery and closures presented with the elastic compression regions. The closures can include a fastening mechanism for selectively closing the compression regions, such that the dimensions of the compression regions can be selectively varied by said selectively varying positioning of the fastening mechanism relative to the compression regions.

### FIELD OF THE INVENTION

The present invention relates generally to post-surgery recovery devices. More particularly, the present invention relates to post-surgery compression devices and methods for use in recovery.

### BACKGROUND

Fat deposits in some people can persist at some body locations despite efforts at dieting or exercising. In such circumstances, cosmetic surgery can be used to remove such fat deposits. Examples of such surgery include liposuction, lipoplasty, liposculpture, and lipectomy, which involve the removal of bodily fat drawn through a suction cannula inserted into an incision in the body.

Once the cosmetic surgery has been completed, the body location subjected to the surgery can swell in size as fluids accumulate in the body tissue. A reduction in such swelling is desirable for optimal results. Physicians have prescribed the use of various compression wraps to apply compressive forces to counter such swelling and fluid accumulation. However, these garments can be uncomfortable and/or ineffective at achieving the new contour and forcing out possible harmful fluids. In addition, such devices do not account for a reduction in swelling over time as the body heals.

Thus, there is a need for improved post-surgery compression devices for use in recovery following surgeries.

### BRIEF SUMMARY

The devices and methods according to the various embodiments of the present invention provide effective post-surgery compression devices and methods for use in recovery following cosmetic surgery, such as liposuction, lipoplasty, abdominoplasty, abdominal contouring, extended abdominal contouring, beltlift for compression of the abdominal areas, after abdominal liposuction, liposculpture, lipectomy, and after revisions of previous abdominal surgeries by including a mechanism for dynamically adjusting the fit of the wrap or garment at selective locations as the body heals.

The device may be worn continuously in the above-referenced procedures for as long as 3 to 5 days and would not be removed in most instances to reduce the amount of bruising, edema, and also reduce the chance of infection developing. After the first 3 to 5 days, the device can be removed to shower, but can sill be worn continuously for a total of 2 weeks. At this point, the device can be used for an additional 2 to 4 weeks, depending on the amount of swelling that is present.

In an embodiment, a compression wrap for use by a user for post-cosmetic surgery recovery includes a first stretchable layer having a length and a height and included of at least two elongated panels, the length being greater than the widest portion of a user's torso, a second stretchable layer selectively coupled to the first layer to define one or more pockets therebetween, one or more resilient panels selectively presented intermediate the first layer and the second layer in the one or more pockets, and a fastening mechanism presented proximate a first end of the first stretchable layer for selectively coupling to a coupling position proximate a second end of the first stretchable layer to form a closed loop, wherein dimensions of the closed loop can be varied by the selectively varying the coupling position proximate the second end.

In another embodiment, a compression garment for use by a user for post-cosmetic surgery recovery includes an upper body portion configured to be worn on an upper body of a user, one or more elastic compression regions shaped to selectively conform to a portion of a user's body subjected to cosmetic surgery, at least one of the one or more elastic compression regions presented on the upper body portion, the one or more elastic compression regions including dimensions greater than dimensions of the portion of the user's body subjected to cosmetic surgery, and one or more closures presented with each of the elastic compression regions, the closures including a fastening mechanism for selectively closing the compression regions, wherein dimensions of the compression regions can be selectively varied by the selectively varying positioning of the fastening mechanism relative to the compression regions.

In a further embodiment, a head wrap for use by a user for post-cosmetic surgery recovery includes a first stretchable fabric region, a second stretchable fabric region, an elastic compression region presented intermediate the first and second stretchable fabric region, and a fastening mechanism presented proximate an end of the first stretchable fabric region opposed the elastic compression region for selectively coupling to a coupling position proximate an end of the second stretchable fabric region opposed the elastic compression region to form a closed loop, wherein dimensions of the closed loop can be varied by the selectively varying the coupling position.

### BRIEF DESCRIPTION OF THE DRAWINGS

The embodiments of the present invention may be more completely understood in consideration of the following detailed description of various embodiments of the invention in connection with the accompanying drawings, in which:

FIG. 1 is a front elevational view depicting a compression wrap according to a first embodiment presented on the lower torso of a person;

FIG. 2 is an elevational view of the compression wrap of FIG. 1, depicting outer, inner, and intermediate layers;

FIG. 3 is an outside elevational view of the compression wrap of FIG. 1;

FIG. 4 is an inside elevational view of the compression wrap of FIG. 1;

FIG. 5 is a cross-sectional schematic view of the outer, inner, and intermediate layers of the compression wrap of FIG. 1;

FIG. 6 is a cross-sectional schematic view of layers of the compression wrap of another embodiment;

FIG. 7 is a front elevational view depicting an upper body compression garment according to a first embodiment presented on the upper body of a female;

FIG. 8 is a rear elevational view of the compression garment of FIG. 7;

FIG. 9 is a front elevational view depicting an upper body compression garment according to a second embodiment presented on the upper body of a female;

FIG. 10 is a rear elevational view of the compression garment of FIG. 9;

FIG. 11 is a front elevational view depicting an upper body compression garment according to a third embodiment presented on the upper body of a male;

FIG. 12 is a front elevational view depicting an upper body compression garment according to a fourth embodiment presented on the upper body of a male;

FIG. 13 is a front elevational view depicting a lower body compression garment according to a first embodiment on the upper legs of a male or female;

FIG. 14 is a front elevational view depicting an upper and lower body compression garment comprising the upper body compression garment of FIG. 7 and the lower body compression garment of FIG. 13 presented on the upper body and upper legs, respectively, of a female;

FIG. 15 is a rear elevational view of the compression garment of FIG. 14;

FIG. 16 is a front elevational view depicting an upper body compression garment according to a fourth embodiment and the lower body compression garment of FIG. 13 on the upper body and upper legs of a male or female;

FIG. 17 is a front elevational view depicting a one-piece upper and lower body compression garment according to a first embodiment presented on the upper body and upper legs of a male;

FIG. 18 is a rear elevational view of the compression garment of FIG. 17;

FIG. 19 is a front elevational view depicting a one-piece upper and lower body compression garment according to a second embodiment presented on the upper body and upper legs of a male;

FIG. 20 is a rear elevational view of the compression garment of FIG. 19;

FIG. 21 is plan view of a head compression garment according to a first embodiment;

FIG. 22 is a side elevational view of the head compression garment of FIG. 21 on the head of a male or female; and

FIG. 23 is an elevational view of a head compression garment according to a second embodiment.

While the embodiments of the present invention are amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the present invention.

### DETAILED DESCRIPTION OF THE DRAWINGS

Compression wraps, garments, and methods of using such wraps and garments can be used to apply compressive forces to counter swelling, minimize brusing and reduce fluid accumulation following various types of cosmetic surgery, such as liposuction, lipoplasty, liposculpture, and lipectomy. Such wraps and garment can include comfort and enable dynamic achievement of a desired body contour and forcing out possible harmful fluids and minimize bruising.

### Compression Wrap

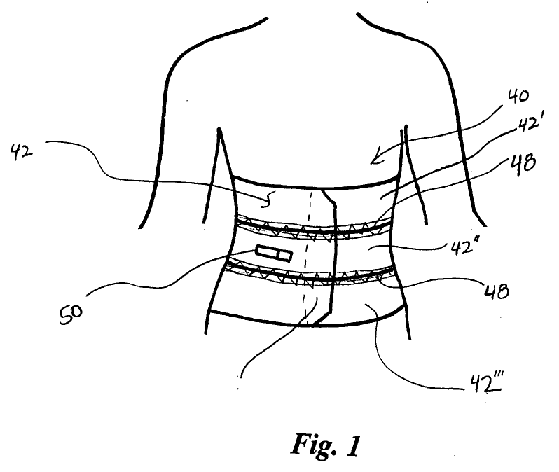

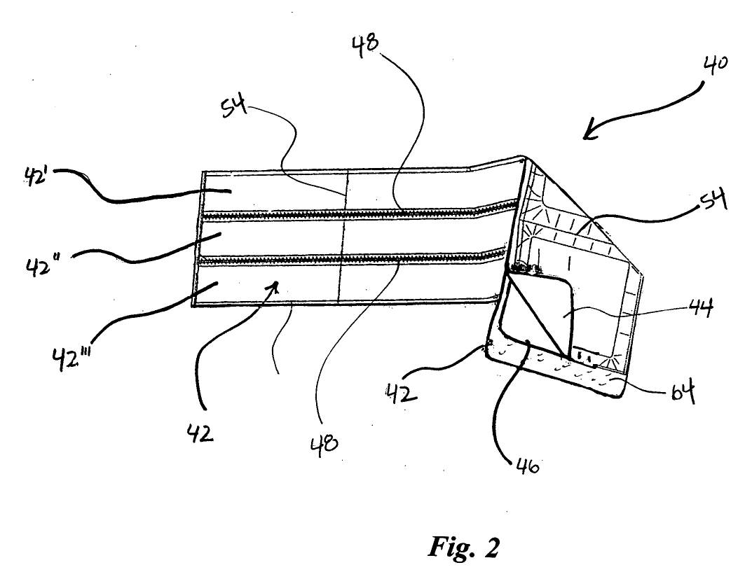

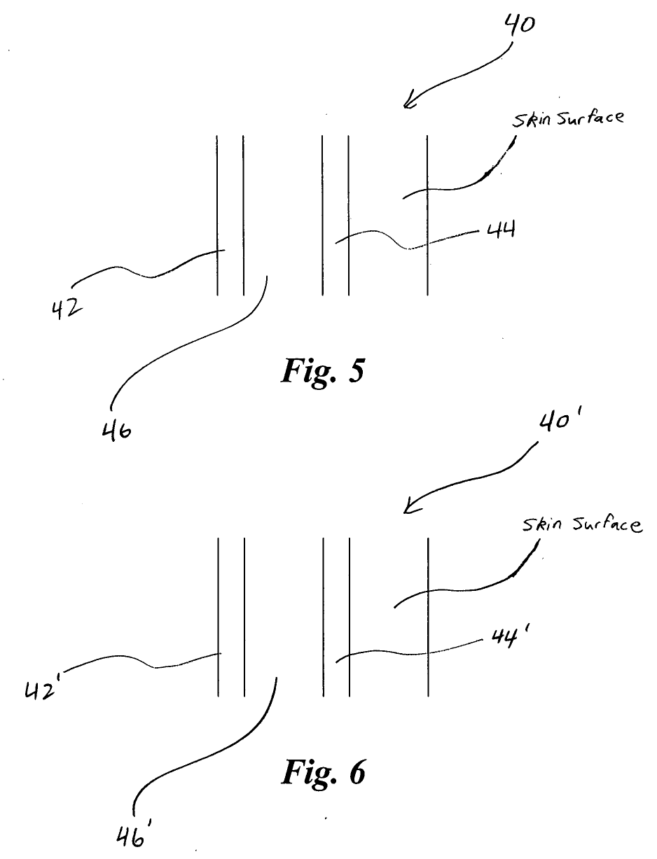

Referring to FIGS. 1 and 2, in a first embodiment, a compression wrap 40 for use following certain types of cosmetic surgery, such as liposuction, lipoplasty, liposculpture, and lipectomy, can broadly include a first, outer layer 42, a second, inner layer 44 for contact with the skin, and an intermediate, resilient layer 46 selectively placed between the intermediate outer and inner layers (FIG. 2). Outer, inner, and intermediate layers 42, 44, 46 can each comprise sub-layers without departing from the scope and spirit of the present disclosure. Layers in addition to first, second, and intermediate layers 42, 44, 46 can also be used without departing from the scope and spirit of the present disclosure. Wrap 40 can further include a hook, loop, or other structure 50 for operably retaining one or more bodily fluid drainage tubes on the outside of wrap 10 (bodily fluid drainage tubes not depicted in the figures). A cross-section of wrap 10 according to the first embodiment is depicted in FIG. 5.

Referring to FIGS. 1-3, outer layer 42 can comprise one or more elongate panels 42′, 42″, 42′″ operably or integrally coupled, such as with stitching 48. As depicted, outer layer 42 comprises three panels 42′, 42″, 42′″ coupled with zigzag stitching 48. The stitching includes a double or triple thread utilizing a hem or interlock style stitch style. Further use of a Surger machine would enforce the strength of the thread. Thread used for stitching 48 between each of panels 42′, 42″, 42′″ can comprise some elasticity and/or slack therein to enable the coupled panels 42′, 42″, 42′″ to move relative to one another to further aid wrap 40 in conforming to the body of a user. Such thread can include nylon, mercerized cotton or a cotton/polyester. In another embodiment, elastic thread can be used. Such configuration also enables wrap 40 to firmly compress and conform to a body as the body shrinks or changes during recovery. Outer layer 42 can be an elastic material, such as expandable fabric. Many elastic fabrics are a non roll polyester/rubber combination. Others may add cotton for a softer feel.

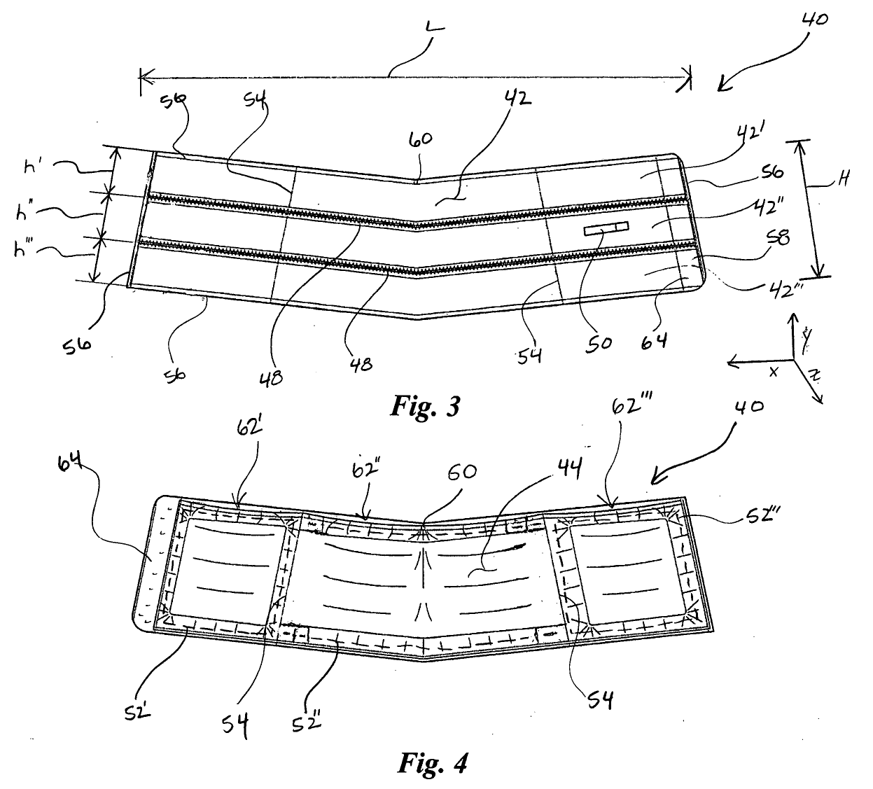

Referring to FIG. 3, wrap 40 comprises a wrap length L and a wrap height H. Panels 42′, 42″, 42′″ can each comprise, for example, individual panel heights h′, h″, h′″ of about three inches, about four inches, or about five inches to collectively form wrap height H, although panel heights of less than about three inches and more than about five inches can be used. When three such panels 42′, 42″, 42′″ are operably coupled (i.e., panels of three, four, or five inches), wrap 40 comprises height H of nine inches, twelve inches, and fifteen inches, respectively, although less than three panels can be used and more than three panels can be used. Also, in embodiments, panels 42′, 42″, 42′″ comprising different heights can be combined and mixed to form wraps of different heights (e.g., two panels of three inches combined with one panel of five inches to form wrap having height of eleven inches). Such combination or mixing can further enable wrap 40 to conform to shape of a user's body.

Referring to FIGS. 2 and 4, inner layer 44 can comprise a fabric blend, such as a cotton/polyester/spandex blend. A desirable feature of inner layer 44 is that it comprises comfort, as it can be in direct contact with the skin. Inner layer 44 can be latex-free and adhesive-free to inhibit any allergic reactions and skin irritations for those that might have allergies to such materials. In an embodiment, inner layer 44 comprises a fabric blend of cotton/polyester/spandex in percentages of about 70% polyester, about 20% cotton, and about 10% spandex. The inner layer may also be made of polyester interlock, nylon/lycra, cotton interlock, two-way stretch polyester/spandex or a closed lycra mesh. Referring to FIG. 3, in embodiments, inner layer 44 can comprise stretchability in all directions relative to x-y axes, in addition to z axis. In other embodiments, inner layer 44 can comprise stretchability in at least four directions (two directions generally parallel to wrap length L along x axis and two directions generally parallel to wrap height H along y axis, in addition to along z axis). In a preferred embodiment, the stretchability of the inner fabric is equal that of the outer.

Referring to FIG. 2, as described above, intermediate layer 46 can comprise one or more resilient panels 52 (depicted as 52′, 52″, 52′″ in FIG. 4). In an embodiment, intermediate panels 52 can comprise a visco-elastic polyurethane or “memory type” foam. Such foam can comprise firmness/gentle combination to maximize contour and comfort. Indentation Force Deflection (“IFD”) ratings for the intermediate panels 52 can range between super soft (about 10 IFD) and semi-rigid (about 120 IFD). Preferably, IFD ratings for the intermediate panels 52 can range from about 12 IFD to about 16 IFD. Thickness can be from ⅛ to 1 inch thick. I prefer ¼ inch thick. The foam actually enhances contour and minimizes brusing. Panels 52 can comprise a width of about a one-quarter inch, although in embodiments, other thicknesses, such as one-eighth inch, one-half inch, and one inch, can be used.

Referring to FIGS. 2 and 4, outer layer 42 and inner layer 44 can be coupled, such as with stitching, proximate and along a selective margins 56 thereof. Each of these margins can include a finish, such as a fabric or other border, to protect the stitching and also provide aesthetic appeal. Other finishes can include, for example, a whipstitch or over sewn edge.

Referring to FIGS. 2 and 4, wrap 40, such as outer layer 42, can comprise a portion of a hook and loop fastening mechanism 64, such as VELCRO®, on an surface thereof, such as an inner surface, presented proximate at least one the margins. As will be described below, hook and loop fastening mechanism 64 can be configured for selective coupling with an outer surface of outer layer 42 or with a complementary portion of a hook and loop fastening mechanism presented on outer layer 42 or on other portion of wrap 40. Other fastening mechanisms can be used in addition to or in lieu of hook and loop fastening mechanism 64 include snaps, hooks, buttons, and zippers.

Outer layer 42 and inner layer 44 can be further coupled, such as with stitching 54, at selective positions transverse to and along wrap length L of wrap to form pockets 62 (depicted as 62′, 62″, 62′″ in FIG. 4) therebetween for the selective placement of intermediate layers panels 52. Intermediate panels 52 can be positioned in the one or more pockets 62 formed between inner and outer layers 44, 42. In an embodiment, such as depicted in FIG. 4, a first panel 52′ is positioned in first pocket 62′, a second intermediate panel 52″ is positioned in second pocket 62″, and a third intermediate panel 52′″ is positioned in third pocket 62′″ (intermediate panels depicted in phantom lines in FIG. 4). In this embodiment, first and third panels 52′, 52′″ are generally shorter in length than second panel 52″, although first, second, and third panels 52′, 52″, 52′″ can be generally equal in length. In the embodiment depicted in FIG. 4, wrap can comprise a snap 60 positioned centrally on second pocket 62″ proximate a top margin of wrap to inhibit opening of second pocket 62″. In another embodiment not depicted, second pocket can be divided into two pockets such that wrap comprises first, second, third, and fourth panels generally equal in length.

Each of the layers of wrap, including inner layer, can comprise an antimicrobial, antibacterial, or bactericidal feature, such as silver-based SILVERtec™ by AccuMED Technologies, Inc. Such features can inhibit or decrease bacteria that can otherwise be present with long-term use of wraps.

Referring again to FIG. 1, in use, wrap 40 can be wrapped around the body region undergoing surgery, such as the abdominal region, such that inner layer 44 is oriented towards skin and outer layer 44 is oriented outwardly away from skin. Hook and loop fastening mechanism 64 can then be selectively coupled with outer layer 42 such that a desired compression is placed around said body region. If the compression becomes too loose or too tight during use, wrap 40 can be readjusted using hook and loop fastening mechanism 64 until a desired compression is re-established.

To provide good support and relatively large compressive forces of wrap 40 on the user when tightly wrapped about a portion of the user, it can be desirable that the stretchability of outer layer panels 42′, 42″, 42′″ be relatively small along the lengths thereof and significantly smaller across the heights thereof. This can enable a tight wrap 40 to remain tight during use under substantial resilient tension but with some lengthwise yield to enable the user to alter bodily configurations through bending with a degree of comfort. At the same time, there can be relatively minimal yielding across the height h′, h″, h′″ of panels 42′, 42″, 42′″ to thereby reduce the resulting compressive forces, a direction in which there is little tension applied during the wrapping of wrap 40 about the user. Differences in girth at different locations along the length of the user's body, locations that are covered by wrap 40 following the wrapping thereof about the user, and bodily bending along that length, can be accommodated by the slack provided in stitching 48.

Wrap 40 with foam intermediate panels 42′, 42″, 42′″ can be used, for example, for upper and lower abdominal compression after liposuction or abdominoplasty. Wrap 40 enables good fit on a body and easy application combining strong compression, smooth contour, and maximum support to surgical areas. Wrap 40 with foam intermediate panels 42′, 42″, 42′″ can improve circulation, minimize swelling and bruising, enable the flushing out of harmful fluids, and accelerate healing.

In an embodiment, wrap 40 for alternative or supplemental use before or after wrap 40 described above is used, can be constructed of material, such as BREATH-O-PRENE® by AccuMED Technologies, Inc. BREATH-O-PRENE® utilizes an open-cell foam layer to maximize breathability and move moisture away from the skin, leaving a body cooler and dryer. BREATH-O-PRENE® can comprise an antimicrobial, antibacterial, or bactericidal feature, such as silver-based SILVERtec™, also by AccuMED Technologies, Inc. Such features can inhibit or eliminate bacteria that can otherwise be present with long-term use of wraps. The wrap according to this embodiment can be constructed of one or more elongate panels operably coupled along a length thereof, such as with stitching, to form wrap.

A cross-section of a wrap 40′ according to this embodiment is depicted in FIG. 6, comprising an inner layer 44′, an open cell polyurethane layer 46′, and an outer layer 42′. Inner layer 44′ can comprise material, such as HOLOFIBER®, which can reflect energy emitted by the body back towards skin to increase oxygen levels, and thus healing. Wrap 40′ according to this embodiment can be constructed of one or more elongate panels operably coupled along a length thereof to form wrap.

### Upper Body Compression Garment—Example One

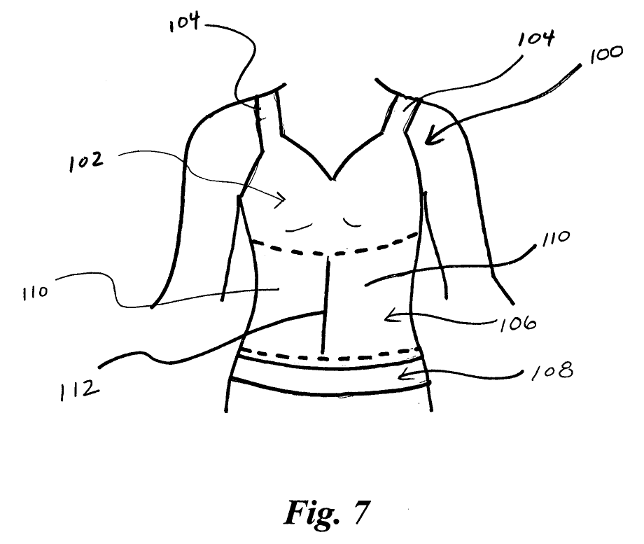

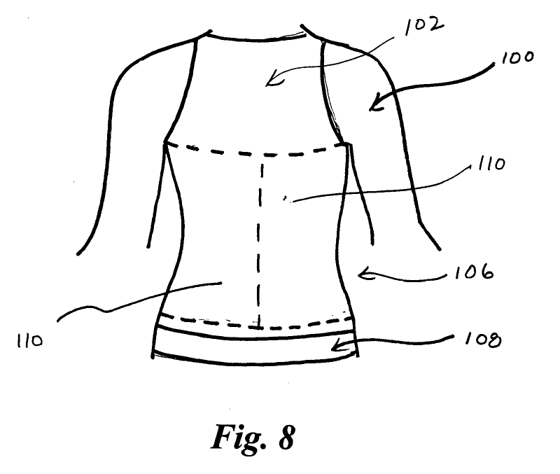

Referring to FIGS. 7 and 8, an upper body garment 100 according to a first embodiment broadly includes an upper portion 102 comprising adjustable shoulder straps 104, a middle, compression portion 106, and a lower portion 108, which can be used for optional coupling with a lower body garment, which is described below.

Middle, compression portion 106 can include one or more pockets 110 for insertion of foam members and a hook and loop fastening mechanism 112, such as VELCRO®, along a height thereof. The inclusion of hook and loop fastening mechanism 112 on upper body garment 100 can provide a mechanism for adjusting wrap contour at selective locations for improved circulation and minimized swelling and accelerated healing. Hook and loop fastening mechanism 112 can thus provide for adequate compression as the swelling diminishes.

In this embodiment, compression portion 106 can be constructed of a polyester/spandex blend. A lightweight polyester/spandex blend can be used for inner pockets 110 to support compression pads (not depicted). Upper portion can comprise a stretch cotton/polyester/spandex blend to add aesthetic appeal at the shoulder and chest area. Lower portion can comprise an elastic waistband attachable to a portion of a hook and loop fastening mechanism at the top of a lower body garment, such as those described below.

Each of the portions of upper body garment 100 can comprise an antimicrobial, antibacterial, or bactericidal feature, such as silver-based SILVERtec™ by AccuMED Technologies, Inc. Such features can inhibit or decrease bacteria that can otherwise be present with long-term use of wraps.

The above-described garment can be useful for surgeries of the abdomen and liposuction of the inner thigh or outer thigh area at the same time. It could also be utilized in cases where liposuction of the lateral chest wall, flank, and back area is accomplished at the same time that surgery of the abdomen and inner thigh region is performed. The two-piece garment can be worn continuously for the first 5 days. Uniform compression is useful to reduce edema, bruising, and reduce the chance of infection developing. After the first 5 days, the two-piece garment can be worn for up to a total of 2 weeks, 24 hours a day, except it could be removed for showering and for washing of the garment. The garments of the present invention can be washed and air dried. After 2 weeks, the garment could be utilized for an additional 4 weeks.

In accordance with an alternative embodiment of the invention, a long two-piece garment extends down to the ankles. In yet another alternative embodiment, the long two-piece garment extends down to the toes. This can be useful in any surgery that involves the chest, back, flank, and includes the thighs, calves, and ankles. It is also useful in surgeries that involve the thighs, calves, and ankles separately. In this instance, the garment can be worn continuously for up to 5 days to reduce edema, bruising, and reduce the chance of infection developing. The garments of the present invention can be washed and air dried. After 2 weeks, the garment could be utilized for an additional 4 weeks.

### Upper Body Compression Garment—Example Two

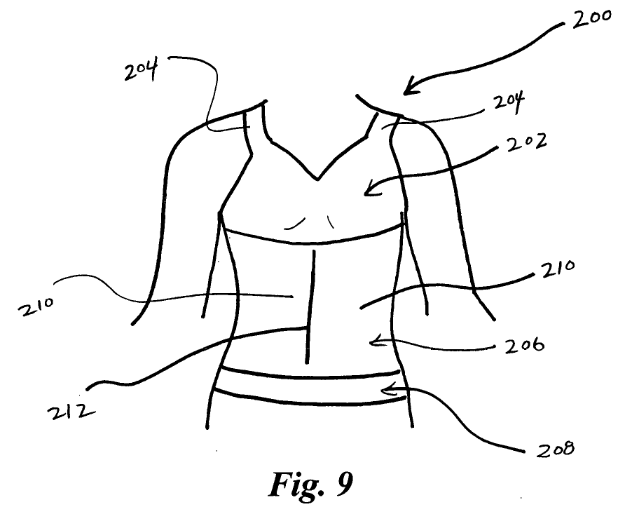

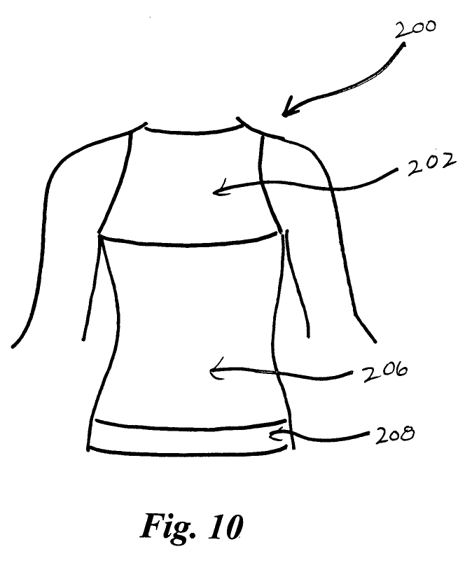

Referring to FIGS. 9 and 10, an upper body garment 200 according to a second embodiment broadly includes an upper portion 202, middle, compression portion 206, and a lower portion 208 for optional coupling with a lower body garment, which is described below. Middle, compression portion 206 can include a hook and loop fastening mechanism 212, such as VELCRO®, along a height thereof. The inclusion of hook and loop fastening mechanism 212 on upper body garment 200 can provide a mechanism for adjusting wrap contour at selective locations for improved circulation and minimized swelling and accelerated healing. Hook and loop fastening mechanism 212 can thus provide for adequate compression as the swelling diminishes.

Middle, compression portion 206 can comprise an open-cell material, such as BREATH-O-PRENE®, which can provide comfort and support for a user approximately at two weeks postoperative. As described above, BREATH-O-PRENE® can improve oxygen delivery to the skin, which, in-turn, can improves circulation and promote healing of the wounds.

Each of the portions of upper body garment can comprise an antimicrobial, antibacterial, or bactericidal feature, such as silver-based SILVERtec™ by AccuMED Technologies, Inc. Such features can inhibit or decrease bacteria that can otherwise be present with long-term use of wraps.

### Upper Body Compression Garment—Example Three

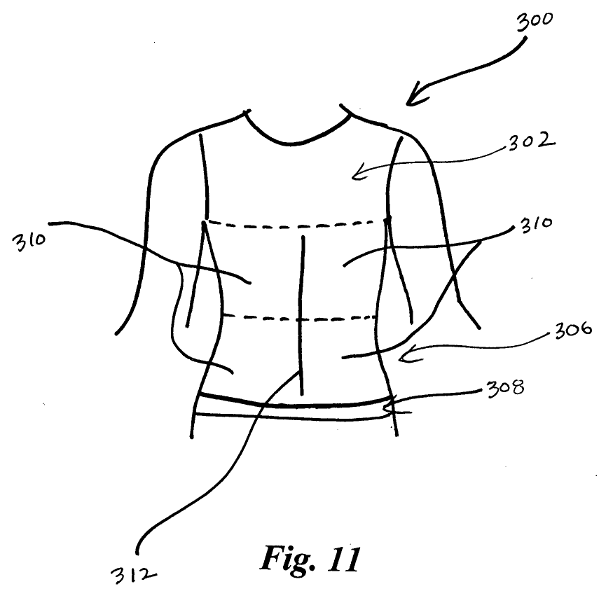

Referring to FIG. 11, an upper body garment 300 according to a third embodiment broadly includes an upper portion 302, middle, compression portion 306, and a lower portion 308 for optional coupling with a lower body garment, which is described below.

Middle, compression portion 306 can include one or more pockets 310 for insertion of foam members and a hook and loop fastening mechanism 312, such as VELCRO®, along a height thereof. The inclusion of hook and loop fastening mechanism 312 on upper body garment 300 can provide a mechanism for adjusting wrap contour at selective locations for improved circulation, minimized swelling, decreased bruising and accelerated healing. Hook and loop fastening mechanism 312 can thus provide for adequate compression as the swelling diminishes.

Upper body garment 300 can maximize compression of the abdomen, chest, and flank areas. Upper body garment 300 can further improve circulation, minimize swelling and bruising, enable the flushing out of harmful fluids, enable smoother contours, and accelerate healing.

Each of the portions of upper body garment 300 can comprise an antimicrobial, antibacterial, or bactericidal feature, such as silver-based SILVERtec™ by AccuMED Technologies, Inc. Such features can inhibit or eliminate bacteria that can otherwise be present with long-term used of wraps.

### Upper Body Compression Garment—Example Four

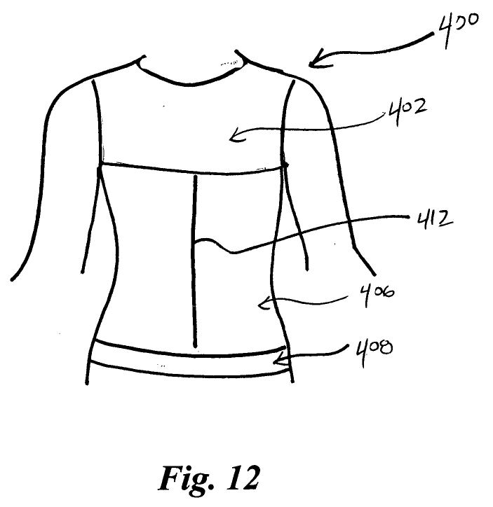

Referring to FIG. 12, an upper body garment 400 according to a fourth embodiment broadly includes an upper portion 402, middle, compression portion 406, and a lower portion 408 for optional coupling with a lower body garment, which will be described below.

Middle, compression portion 406 can include a hook and loop fastening mechanism 412, such as VELCRO®, along a height thereof. The inclusion of hook and loop fastening mechanism 412 on upper body garment can provide a mechanism for adjusting wrap contour at selective locations for improved circulation and minimized swelling and accelerated healing. Hook and loop fastening mechanism 412 can thus provide for adequate compression as the swelling diminishes.

Middle, compression portion 406 can comprise an open-cell material, such as BREATH-O-PRENE®, which can provide comfort and support for a user approximately at two weeks postoperative. As described above, BREATH-O-PRENE® can improve oxygen delivery to the skin, which, in-turn, can improve circulation and promote healing of the wounds.

Each of the portions of upper body garment 400 can comprise an antimicrobial, antibacterial, or bactericidal feature, such as silver-based SILVERtec™ by AccuMED Technologies, Inc. Such features can inhibit or decrease bacteria that can otherwise be present with long-term use of wraps.

### Lower Body Compression Garment—Example One

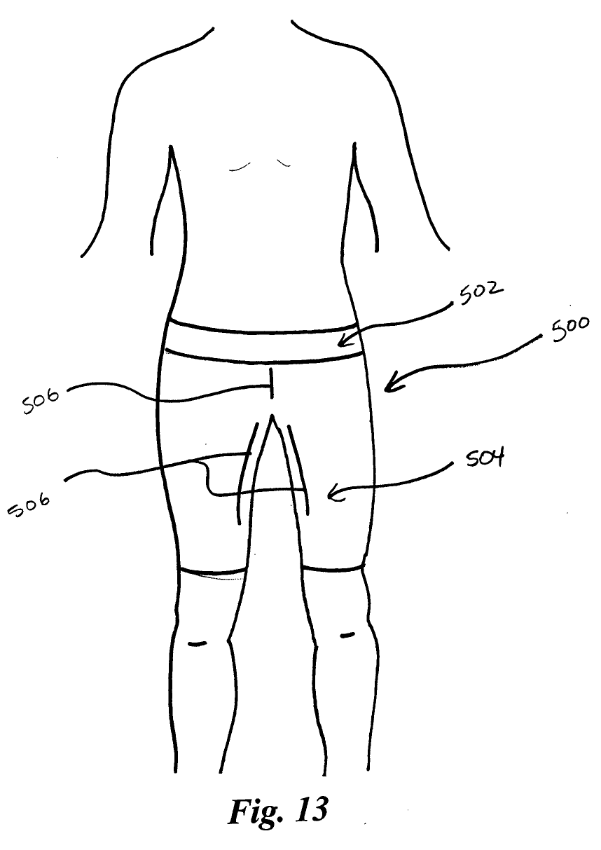

Referring to FIG. 13, a lower body garment 500 according to a first embodiment broadly includes an upper portion 502 for optional coupling with an upper body garment, such as those described above, and a lower compression portion 504. Lower, compression portion 504 can include one or more hook and loop fastening mechanisms 506, such as VELCRO®. The inclusion of hook and loop fastening mechanisms 506 on lower body garment 500 can provide a mechanism for adjusting wrap contour at selective locations for improved circulation and minimized swelling and accelerated healing. Hook and loop fastening mechanisms 506 can thus provide for adequate compression as the swelling diminishes.

Each of the portions of the lower body garment 500 can comprise an antimicrobial, antibacterial, or bactericidal feature, such as silver-based SILVERtec™ by AccuMED Technologies, Inc. Such features can inhibit or decrease bacteria that can otherwise be present with long-term use of wraps.

### Upper and Lower Body Compression Garment—Example One

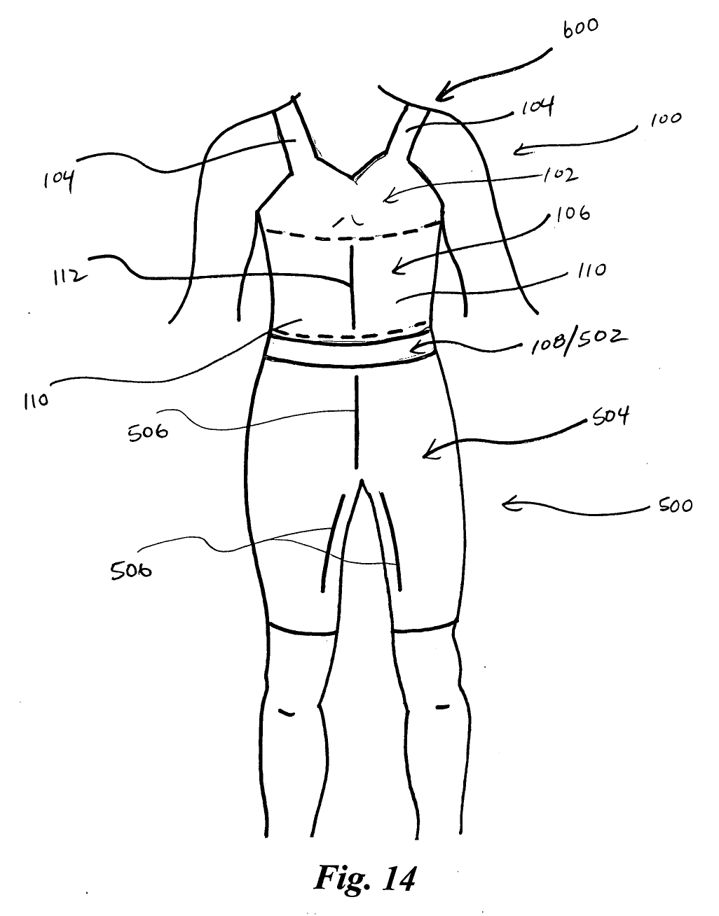

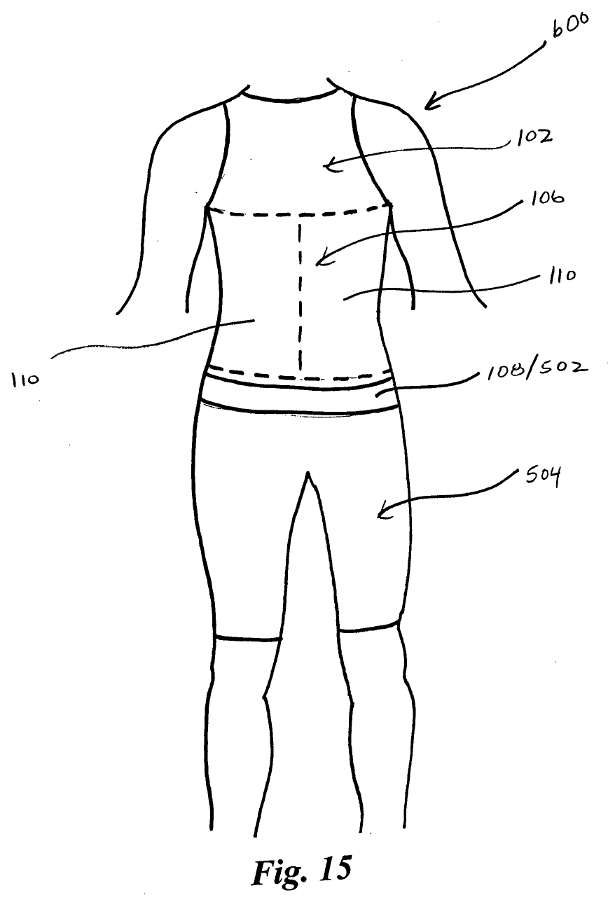

Referring to FIG. 14, an upper and lower body compression garment 600 according to a first embodiment comprises an upper body compression garment 100 and a lower body compression garment 500. As depicted in FIG. 14, upper and lower body compression garment 600 comprises upper body compression garment 100 of FIG. 7 and lower body compression garment 500 of FIG. 13 presented on the upper body and upper legs, respectively.

Compression garment 600 can be used for upper and lower abdominal, back, flanks, hips, and thigh compression after liposuction or abdominoplasty. Compression garment 600 is depicted as a two-piece garment that can be attached at the waist with the one-sided open loop hook and loop fastening mechanism. Compression garment 600 can be adjustable at the abdomen, both upper and lower. Compression garment 600 also includes hook and loop fastening mechanisms 506 at the inner leg.

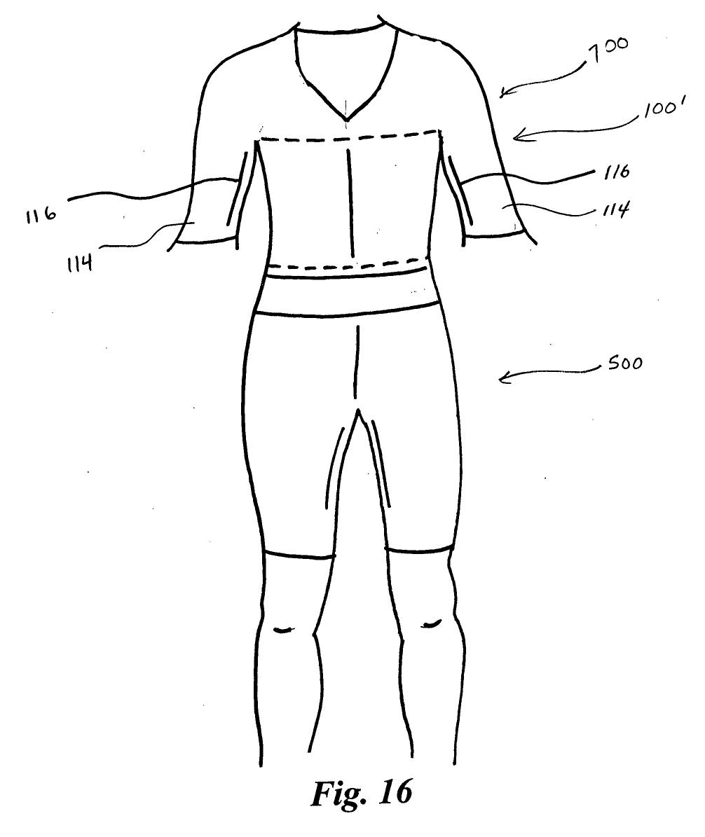

Referring to FIG. 16, in another embodiment, upper body portion 100′ can further comprise partial or full sleeves 114 including one or more hook and loop fastening mechanisms 116, such as VELCRO®. The inclusion of hook and loop fastening mechanisms can provide a mechanism for adjusting wrap contour at selective locations for improved circulation and minimized swelling and accelerated healing. Hook and loop fastening mechanisms 116 can thus provide for adequate compression as the swelling diminishes.

Each of the portions of the upper and lower body compression garment can comprise an antimicrobial, antibacterial, or bactericidal feature, such as silver-based SILVERtec™ by AccuMED Technologies, Inc. Such features can inhibit or decrease bacteria that can otherwise be present with long-term use of wraps.

### Upper and Lower Body Compression Garment—Example Three

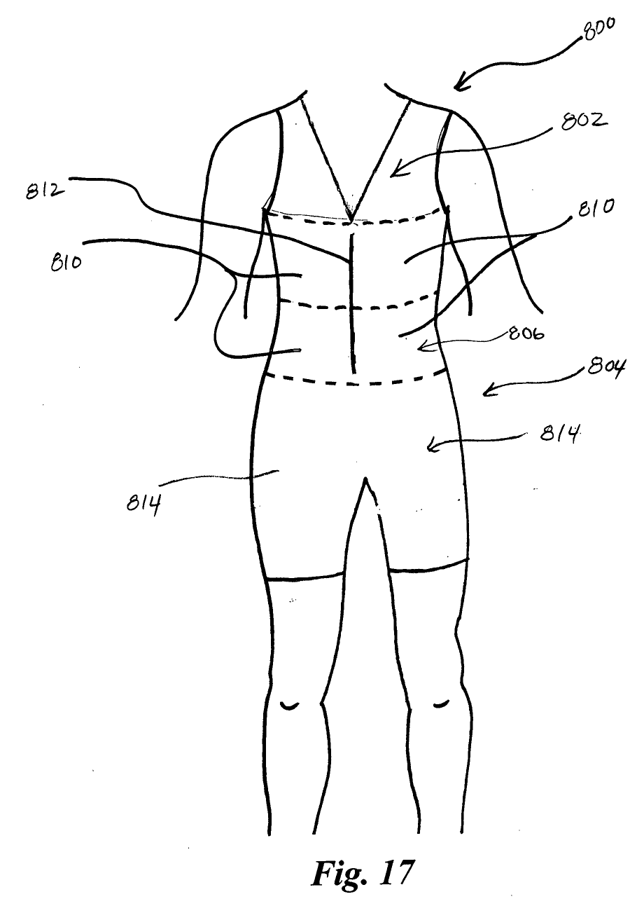

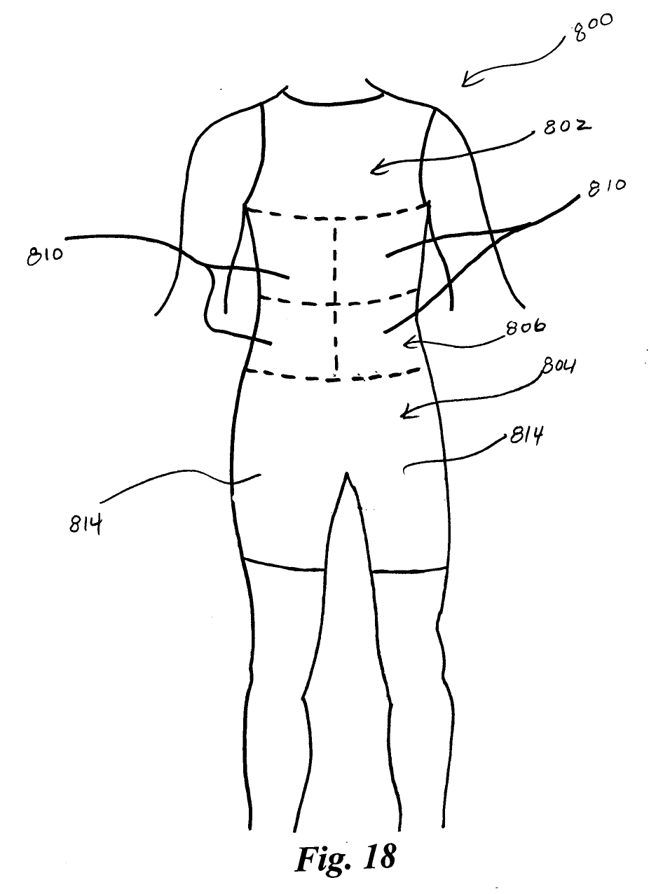

Referring to FIGS. 17 and 18, an upper and lower body compression garment 800 according to a third embodiment comprises an upper body portion 802 and a lower body portion 804.

Lower body portion 804 can comprise first and second partial or full leg portions 814. As depicted, lower body portion 804 comprises partial leg or short-like portions 814.

Upper body portion 802 comprises an upper portion 802 and middle, compression portion 806. Middle, compression portion 806 can include one or more pockets 810 for insertion of foam members and a hook and loop fastening mechanism 812, such as VELCRO®, along a height thereof. The inclusion of hook and loop fastening mechanism 812 on upper body portion 802 can provide a mechanism for adjusting abdominal wrap contour for improved circulation and minimized swelling and accelerated healing. Hook and loop fastening mechanism 812 can thus provide for adequate compression as the swelling diminishes. Referring to FIGS. 17 and 18, upper body portion comprises four pockets on a front side thereof (FIG. 17) and four pockets on a rear side thereof (FIG. 18).

Each of the portions of the upper and lower body compression garment can comprise an antimicrobial, antibacterial, or bactericidal feature, such as silver-based SILVERtec™ by AccuMED Technologies, Inc. Such features can inhibit or decrease bacteria that can otherwise be present with long-term use of wraps.

### Upper and Lower Body Compression Garment—Example Four

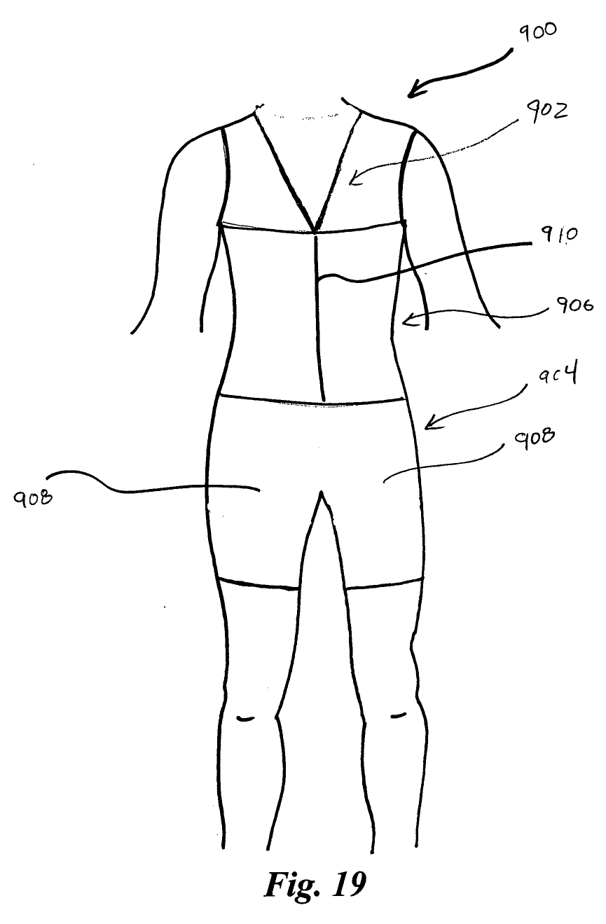

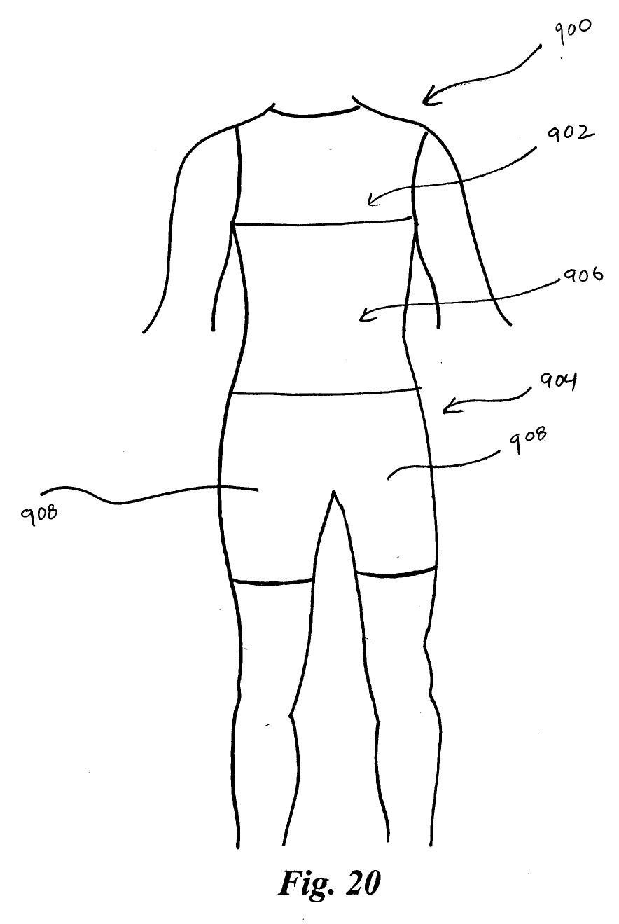

Referring to FIG. 19, an upper and lower body compression garment 900 according to a third embodiment comprises an upper body portion 902 and a lower body portion 904.

Lower body portion 904 can comprise first and second partial or full leg portions 908. As depicted, lower body portion 904 comprises partial leg or short-leg portions 908.

Upper body portion 902 comprises a compression portion 906. Compression portion can include a hook and loop fastening mechanism 910, such as VELCRO®, along a height thereof. The inclusion of hook and loop fastening mechanism 910 on upper body garment 900 can provide a mechanism for adjusting wrap contour at selective locations for improved circulation and minimized swelling and accelerated healing. Hook and loop fastening mechanism 910 can thus provide for adequate compression as the swelling diminishes.

Middle, compression portion 910 can comprise an open-cell material, such as BREATH-O-PRENE®, which can provide comfort and support for a user approximately at two weeks postoperative. As described above, BREATH-O-PRENE® can improve oxygen delivery to the skin, which, in-turn, can improves circulation and promote healing of the wounds.

Each of the portions of upper and lower body compression garment can comprise an antimicrobial, antibacterial, or bactericidal feature, such as silver-based SILVERtec™ by AccuMED Technologies, Inc. Such features can inhibit or decrease bacteria that can otherwise be present with long-term use of wraps.

### Head Wrap—Example One

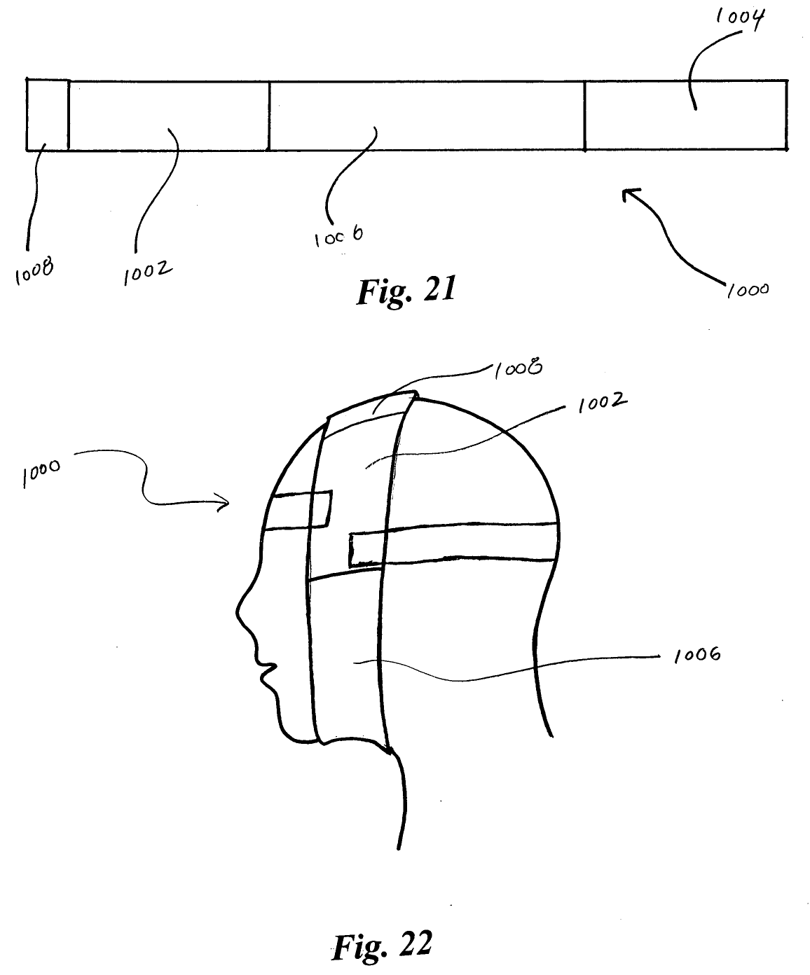

Referring to FIGS. 21 and 22, a head wrap 1000 according to a first embodiment comprises a first fabric portion 1002, a second fabric portion 1004, a section comprised of an open-cell material 1006, such as BREATH-O-PRENE®, and a hook and loop fastening mechanism 1008, such as VELCRO®. First and second fabric portions 1002, 1004 can comprise a medium-weight cotton/polyester/spandex, such as a fabric blend of cotton/polyester/spandex in percentages of about 70% polyester, about 20% cotton, and about 10% spandex. Other fabrics might include a closed cotton/polyester mesh, lycra mesh, cotton/spandex, nylon/lycra or a cotton interlock fabric.

Referring to FIG. 22, head wrap 1000 can be placed about a user's head such that the open-cell material 1006 is positioned proximate the user's chin. In this configuration, head wrap 1000 can be used for by a user who has had a facelift or liposuction of the chin.

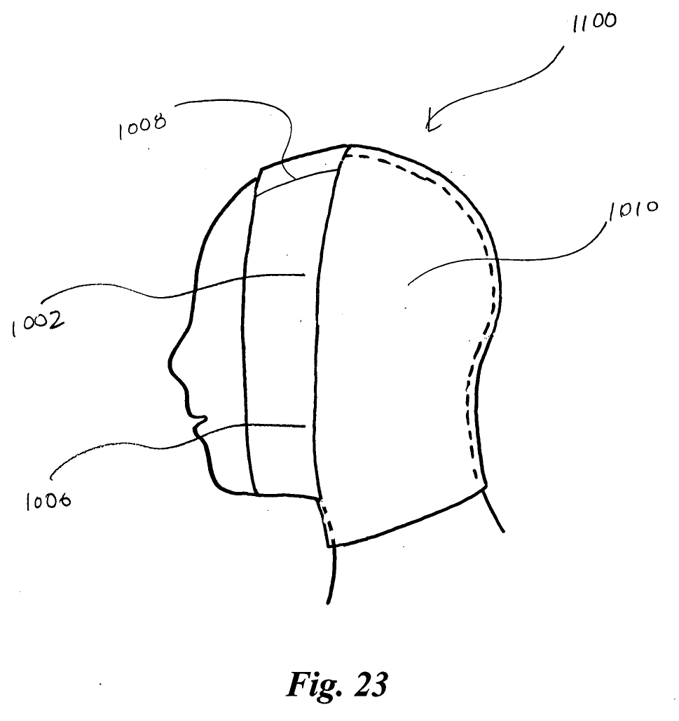

Referring to FIG. 23, in another embodiment, head wrap 1100 comprises a head cover 1010 operably coupled thereto. Head cover 1010 can comprise a medium-weight cotton/polyester/spandex, such as a fabric blend of cotton/polyester/spandex in percentages of about 70% polyester, about 20% cotton, and about 10% spandex. In this embodiment, apertures 1012 can be included on the head cover 1010 corresponding to the user's ears to provide ease of hearing.

Each of the portions of head wrap 1000 can comprise an antimicrobial, antibacterial, or bactericidal feature, such as silver-based SILVERtec™ by AccuMED Technologies, Inc. Such features can inhibit or eliminate bacteria that can otherwise appear with long-term used of wraps.

### Post-Surgery Method—First Example

The wrap compression garment would be applied immediately after the surgical procedure. The garment would be appropriate for:

1. Abdominoplasty (tummy tuck)

2. Abdominal contouring (abdominoplasty with liposuction of the flank and upper abdominal area).

3. Extended abdominal contouring (1 and 2 plus further extension of excision in the lower back and flank area.

4. Beltlift for compression of the abdominal areas, as in examples 1-3.

5. After abdominal liposuction (suction-assisted lipoplasty, liposelection, etc.).

6. After revisions of previous abdominal surgeries.

The wrap would be worn continuously in most of these procedures for as long as 3-5 days and would not be removed in most instances to reduce the amount of bruising, edema, and also reduce the chance of infection developing. After the first 3-5 days, the wrap would be worn continuously for a total of 2 weeks. Patients would be allowed to remove the wrap to shower. After 2 weeks, the BREATH-O-PRENE wrap would be used for an additional 2-4 weeks, depending on the amount of swelling that is present. This would fit better under clothing.

### Post-Surgery Method—Second Example

The device would be appropriate for surgery of the abdomen and liposuction of the inner thigh or outer thigh area at the same time. It could also be utilized in cases where liposuction of the lateral chest wall, flank, and back area is accomplished at the same time that surgery of the abdomen and inner thigh region is performed. The two-piece garment would typically be worn continuously for the first 5 days. Even compression is useful to reduce edema (swelling), bruising, and reduce the chance of infection developing. After the first 5 days, the two-piece garment would be worn for up to a total of 2 weeks, 24 hours a day, except it could be removed for showering and for washing and air-drying of the garment. After 2 weeks, a BREATH-O-PRENE garment could be utilized for an additional 4 weeks.

### Post-Surgery Method—Third Example

The long two-piece garment extends down to the ankles or actually incorporates the feet and extends to the toes. This would be appropriate in any surgery that involves the chest, back, flank, and includes the thighs, calves, and ankles. It would also be appropriate in surgeries that involve the thighs, calves, and ankles separately. The lower half of the garment could be utilized in those instances. Again, it would be worn continuously for up to 5 days to reduce the edema, bruising, and reduce the chance of infection developing. The Stage II garment would be the BREATH-O-PRENE equivalent of the Stage I garment. The Stage II garment is worn for an additional 2-4 weeks.

The embodiments above are intended to be illustrative and not limiting. Additional embodiments are within the claims. In addition, although the present invention has been described with reference to particular embodiments, those skilled in the art will appreciate that changes can be made in form and detail without departing from the spirit and scope. Any incorporation by reference of documents above is limited such that no subject matter is incorporated that is contrary to the explicit disclosure herein.

What is claimed is:

1. A compression wrap for post-cosmetic surgery recovery, said wrap comprising:
a first stretchable layer having a length and a height and comprised of at least two elongate panels, said length being greater than a portion of a user's torso subjected to cosmetic surgery;
a second stretchable layer selectively coupled to the first stretchable layer to define one or more pockets therebetween;
one or more pads selectively presented intermediate said first stretchable layer and said second stretchable layer in said one or more pockets; and
a fastening mechanism presented proximate a first end of said first stretchable layer for selectively coupling to a coupling position proximate a second end of said first stretchable layer to form a closed loop, wherein a circumference of said closed loop can be varied by said selectively varying said coupling position proximate said second end.

2. The wrap of claim 1, wherein the one or more pads comprise polyurethane foam.

3. The wrap of claim 1, wherein the one or more pads comprise visco-elastic polyurethane foam.

4. The wrap of claim 1, further comprising a drainage tube retention mechanism.

5. The wrap of claim 1, further comprising an antimicrobial feature.

6. The wrap of claim 1, further comprising a silver-based antimicrobial feature.

7. The wrap of claim 1, wherein said fastening mechanism comprises a hook and loop fastener.

8. A compression garment for post-cosmetic surgery recovery, said garment comprising:
an upper body portion configured to be worn on an upper body of a user;
one or more elastic compression regions shaped to selectively conform to a portion of a user's body subjected to cosmetic surgery, at least one of said one or more elastic compression regions presented on said upper body portion; and
one or more closures presented with each of said one or more elastic compression regions, said one or more closures comprising a fastening mechanism for selectively closing said elastic compression regions, wherein a dimension of said compression regions can be selectively varied by said selectively varying positioning of said fastening mechanism relative to said compression regions.

9. The garment of claim 8, wherein said fastening mechanism comprises a hook and loop fastener.

10. The garment of claim 8, wherein said compression portion comprises first and second stretchable layers selectively coupled to define one or more pockets therebetween, said garment further comprising one or more pads selectively presented intermediate said first and second stretchable layers in said one or more pockets.

11. The garment of claim 10, wherein the one or more pads comprise polyurethane foam.

12. The garment of claim 10, wherein the one or more pads comprise visco-elastic polyurethane foam.

13. The garment of claim 10, comprising sleeves thereon, wherein at least one of the one or more closures are presented on said sleeves.

14. The garment of claim 10, wherein at least one of the one or more closures is presented on a region of said upper body portion corresponding to said user's abdomen.

15. The garment of claim 10, further comprising a lower body portion configured to be worn on a lower body of said user, said garment further comprising at least two elastic compression regions, wherein at least one of said at least two elastic compression regions is presented on said lower body portion.

16. The garment of claim 8, further comprising a lower body portion configured to be worn on a lower body of said user, said garment further comprising at least two elastic compression regions, wherein at least one of said at least two elastic compression regions is presented on said lower body portion.

17. A head wrap for post-cosmetic surgery recovery, said wrap comprising:
a first stretchable region;
a second stretchable region;
an elastic compression region presented intermediate said first and second stretchable regions; and
a fastening mechanism presented proximate an end of said first stretchable region opposed said elastic compression region for selectively coupling to a coupling position proximate an end of said second stretchable fabric region opposed said elastic compression region to form a closed loop, wherein dimensions of said closed loop can be varied by said selectively varying said coupling position.

18. The wrap of claim 15, wherein said fastening mechanism comprises a hook and loop fastener.

19. The wrap of claim 15, further comprising a head cover coupled along a length of said wrap and configured to conform to a head shape of a user.

20. The wrap of claim 15, further comprising an antimicrobial feature.
